(19) United States
(12) Patent Application Publication
(10) Pub. No.: US 2023/0218867 A1
(43) Pub. Date: Jul. 13, 2023

(54) Introducer Needles, Catheter Insertion Assemblies, and Methods Thereof

(71) Applicant: Bard Access Systems, Inc., Salt Lake City, UT (US)

(72) Inventors: Glade H. Howell, Draper, UT (US); Daniel B. Blanchard, Bountiful, UT (US); Kyle G. Thornley, Farmington, UT (US)

(21) Appl. No.: 18/095,968

(22) Filed: Jan. 11, 2023

Related U.S. Application Data
(60) Provisional application No. 63/298,384, filed on Jan. 11, 2022.

(51) Int. Cl. A61M 25/06; A61M 25/09

(52) U.S. Cl. CPC A61M 25/0606; A61M 25/09

(57) ABSTRACT

An introducer needle can include a needle shaft, a sheath over the needle shaft, and a needle hub around a proximal portion of each of the needle shaft and the sheath. The needle shaft can include a needle slot extending from the proximal portion of the needle shaft through a distal needle tip, which needle tip can include a bevel having a tip bevel and a heel. The needle shaft can include opposite needle-slot walls that either: a) face each other and are parallel or oblique to each other, or b) face toward or away from a bottom of the needle slot. Additionally or alternatively, opposite needle-shaft walls can be parallel to each other. The sheath over the needle shaft can seal the needle slot thereunder but for a sheath opening in a proximal portion of the sheath.

### PRIORITY

This application claims the benefit of priority to U.S. Provisional Application No. 63/298,384, filed Jan. 11, 2022, which is incorporated by reference in its entirety into this application.

### BACKGROUND

Central venous catheters (“CVCs”) are commonly introduced into patients and advanced through their vasculatures by way of the Seldinger technique. The Seldinger technique utilizes a number of steps and medical devices (e.g., a needle, a scalpel, a guidewire, an introducer sheath, a dilator, a CVC, etc.). While the Seldinger technique is effective, the number of steps are time consuming, handling the number of medical devices is awkward, and both of the foregoing can lead to patient trauma. In addition, there is a relatively high potential for touch contamination due to the number of medical devices that need to be interchanged during the Seldinger technique. As such, there is a need to reduce the number of steps and medical devices involved in introducing a catheter such as a CVC into a patient and advancing the catheter through a vasculature thereof

Disclosed herein are introducer needles for insertion assemblies of rapidly insertable central catheters (“RICCs”) and methods that address the foregoing.

### SUMMARY

Disclosed herein is an introducer needle including, in some embodiments, a needle shaft, a sheath over the needle shaft, and a needle hub around both a proximal portion of the needle shaft and a proximal portion of the sheath. The needle shaft includes a needle slot extending from a proximal portion of the needle shaft through a distal needle tip, which needle tip includes a bevel having a tip bevel and a heel. The needle shaft includes one or more features selected from a group of features consisting of: a) opposite needle-slot walls face each other and are parallel to each other consistent with the needle slot being cut into the needle shaft; b) the opposite needle-slot walls face each other but are oblique to each other consistent with the needle shaft being stamped or rolled to form the needle slot; c) the opposite needle-slot walls face away from a bottom of the needle slot consistent with the needle slot being ground into the needle shaft; d) the opposite needle-slot walls face toward the bottom of the needle slot consistent with opposite needle-slot walls being bent in toward the bottom of the needle slot; and e) the opposite needle-shaft walls are parallel to each other consistent with the needle shaft being stamped or rolled to form the needle slot. The sheath over the needle shaft seals the needle slot thereunder but for a sheath opening in a proximal portion of the sheath.

In some embodiments, the needle slot extends along at least a portion of a top of the needle shaft. The top of the needle shaft includes the heel of the bevel.

In some embodiments, the needle slot extends along at least a portion of a bottom of the needle shaft. The bottom of the needle shaft includes the tip bevel of the bevel.

In some embodiments, the needle slot extends along at least a portion of a side of the needle shaft. The side of the needle shaft is between the heel and tip bevel of the bevel.

In some embodiments, the needle slot bisects the heel of the bevel.

In some embodiments, the needle slot bisects the tip bevel of the bevel.

In some embodiments, the needle slot intersects the heel, the tip bevel, or both the heel and the tip bevel of the bevel without bisecting the heel or tip bevel of the bevel.

In some embodiments, the needle slot is linear from the proximal portion of the needle shaft through the needle tip.

In some embodiments, the needle slot is non-linear from the proximal portion of the needle shaft through the needle tip.

In some embodiments, the needle slot is at least partially helical. The needle slot forms at least part of a helix between the proximal portion of the needle shaft and the needle tip.

In some embodiments, the needle slot has a constant needle-slot width.

In some embodiments, the needle slot has a non-constant needle-slot width.

In some embodiments, the needle slot includes one or more needle-slot openings wider than a remainder of the needle slot.

In some embodiments, the needle slot includes a taper, a reverse taper, or a stepwise change in the needle-slot width from the proximal portion of the needle shaft through the needle tip.

In some embodiments, the needle slot is short of a proximal end of the needle

shaft.

In some embodiments, the needle slot extends through a proximal end of the

needle shaft.

In some embodiments, edges of the needle-slot walls are finished. The edges of the needle-slot walls being finished minimizes or eliminates sharp, access guidewire-fraying edges of the needle slot.

Also disclosed herein is a RICC insertion assembly including, in some embodiments, a RICC, an introducer needle, an access guidewire disposed in the RICC and the introducer needle, and a coupler coupling the RICC and the introducer needle together. The RICC includes a catheter tube and a primary lumen therethrough. The introducer needle includes a needle shaft, a sheath over the needle shaft, and a needle hub around both a proximal portion of the needle shaft and a proximal portion of the sheath. The needle shaft includes a needle slot extending from a proximal portion of the needle shaft through a distal needle tip, which needle tip includes a bevel having a tip bevel and a heel. The needle shaft includes one or more features selected from a group of features consisting of: a) opposite needle-slot walls face each other and are parallel to each other consistent with the needle slot being cut into the needle shaft; b) the opposite needle-slot walls face each other but are oblique to each other consistent with the needle shaft being stamped or rolled to form the needle slot; c) the opposite needle-slot walls face away from a bottom of the needle slot consistent with the needle slot being ground into the needle shaft; d) the opposite needle-slot walls face toward the bottom of the needle slot consistent with opposite needle-slot walls being bent in toward the bottom of the needle slot; and e) the opposite needle-shaft walls are parallel to each other consistent with the needle shaft being stamped or rolled to form the needle slot. The sheath over the needle shaft seals the needle slot thereunder but for a sheath opening in a proximal portion of the sheath. The access guidewire includes a proximal portion disposed in the primary lumen of the RICC and a distal portion disposed in the needle shaft through both the sheath opening and needle slot.

In some embodiments, the needle slot extends along at least a portion of a top of the needle shaft. The top of the needle shaft includes the heel of the bevel.

In some embodiments, the needle slot extends along at least a portion of a bottom of the needle shaft. The bottom of the needle shaft includes the tip bevel of the bevel.

In some embodiments, the needle slot extends along at least a portion of a side of the needle shaft. The side of the needle shaft is between the heel and tip bevel of the bevel.

In some embodiments, the needle slot bisects the heel of the bevel.

In some embodiments, the needle slot bisects the tip bevel of the bevel.

In some embodiments, the needle slot intersects the heel, the tip bevel, or both the heel and the tip bevel of the bevel without bisecting the heel or tip bevel of the bevel.

In some embodiments, the needle slot is linear from the proximal portion of the needle shaft through the needle tip.

In some embodiments, the needle slot is non-linear from the proximal portion of the needle shaft through the needle tip.

In some embodiments, the needle slot is at least partially helical. The needle slot forms at least part of a helix between the proximal portion of the needle shaft and the needle tip.

In some embodiments, the needle slot has a constant needle-slot width.

In some embodiments, the needle slot has a non-constant needle-slot width.

In some embodiments, the needle slot includes one or more needle-slot openings wider than a remainder of the needle slot.

In some embodiments, the needle slot includes a taper, a reverse taper, or a stepwise change in the needle-slot width from the proximal portion of the needle shaft through the needle tip.

In some embodiments, the needle slot is short of a proximal end of the needle shaft.

In some embodiments, the needle slot extends through a proximal end of the needle shaft.

In some embodiments, edges of the needle-slot walls are finished. The edges of the needle-slot walls being finished minimizes or eliminates sharp, access guidewire-fraying edges of the needle slot.

In some embodiments, the coupler includes a coupler housing including a sealing-module cavity and a sealing-module insert disposed in the sealing-module cavity. The sealing-module cavity and the sealing-module insert form a sealing module of the RICC insertion assembly. The sealing module is configured to separately seal around a proximal portion of the introducer needle including the sheath opening and the distal portion of the access guidewire when compressed in the sealing-module cavity.

In some embodiments, the coupler housing includes a longitudinal coupler-housing slot. The coupler-housing slot is configured to allow the access guidewire to escape from the coupler housing after a distal portion of the sealing-module insert is removed from the sealing-module cavity with withdrawal of the introducer needle from the coupler.

In some embodiments, the coupler includes a blade extending into the sealing module such that the blade is disposed in the needle slot under a distal end of the sheath opening. The blade includes a distal-facing blade edge configured to cut the sheath away from the needle shaft as the introducer needle is withdrawn from the coupler, thereby allowing the access guidewire to escape from the needle shaft by way of the needle slot.

Also disclosed herein is a method of making an introducer needle. The method includes, in some embodiments, a needle-slot creating step, a sheath-disposing step, and a needle hub-fixing step. The needle-slot creating step includes creating a needle slot in a needle shaft. The needle slot extends from a proximal portion of the needle shaft through a distal needle tip, which needle tip includes a bevel having a tip bevel and a heel. The sheath-disposing step includes disposing a sheath over the needle shaft. The sheath seals the needle slot thereunder. The needle hub-fixing step includes fixing a needle hub around both the proximal portion of the needle shaft and a proximal portion of the sheath, thereby forming the introducer needle.

In some embodiments, the needle-slot creating step includes cutting the needle slot into the needle shaft by way of machining or laser cutting.

In some embodiments, opposite needle-slot walls face each other and are parallel to each other subsequent to the cutting. In addition, the needle-slot walls are opposite a bottom of the needle slot.

In some embodiments, the needle-slot creating step includes grinding the needle slot into the needle shaft.

In some embodiments, opposite needle-slot walls face away from a bottom of the needle slot subsequent to the grinding.

In some embodiments, the method further includes a wall-bending step. The wall-bending step includes bending the needle-slot walls in toward the bottom of the needle slot, thereby increasing a flexural strength of the needle shaft.

In some embodiments, the method further includes an edge-finishing step. The edge-finishing step includes finishing edges of the needle slot walls, thereby minimizing or eliminating sharp, access guidewire-fraying edges of the needle slot.

In some embodiments, the sheath-disposing step includes inserting the needle shaft into the sheath.

In some embodiments, the sheath-disposing step further includes heat shrinking the sheath over the needle shaft.

In some embodiments, the method further includes a sheath opening-creating step. The sheath opening-creating step includes creating a sheath opening in the sheath. The sheath seals the needle slot thereunder but for the sheath opening in a proximal portion of the sheath.

In some embodiments, the sheath opening-creating step includes cutting the sheath opening into the needle shaft by way of laser cutting after disposing the sheath over the needle shaft in the sheath-disposing step.

In some embodiments, the needle hub-fixing step includes adhering the needle hub to the both the proximal portion of the needle shaft and the proximal portion of the sheath.

In some embodiments, the needle hub-fixing step includes pressing both the proximal portion of the needle shaft and the proximal portion of the sheath into the needle hub with an engineering fit selected from a transition and an interference fit.

In some embodiments, the method further includes a metal strip-rolling step, a seam-welding step, a cold-working step, and a grinding step. The metal strip-rolling step includes rolling a strip of metal into a metal tube. The metal tube includes a longitudinal seam formed between edges of longitudinal sides of the strip of metal. The seam-welding step includes welding the seam. The cold-working step includes pushing the metal tube through one or more dies, thereby reducing an outer diameter of the metal tube while simultaneously increasing a thickness of a metal-tube wall of the metal tube. The grinding step includes grinding an end of the metal tube at a plurality of angles to form the needle shaft with the needle tip having the bevel.

These and other features of the concepts provided herein will become more apparent to those of skill in the art in view of the accompanying drawings and following description, which describe particular embodiments of such concepts in greater detail.

### DRAWINGS

FIG. 1 illustrates a RICC insertion assembly in accordance with some embodiments.

FIG. 2 illustrates a perspective view of a coupler of the RICC insertion assembly in accordance with some embodiments.

FIG. 3 illustrates a longitudinal cross section of the coupler and a splittable sealing module thereof in accordance with some embodiments.

FIG. 4 illustrates a longitudinal cross section of the needle hub and the proximal and distal portions of the sealing-module insert in accordance with some embodiments.

FIG. 5 illustrates a top view of the introducer needle in accordance with some embodiments.

FIG. 6 illustrates a sheath of the introducer needle in accordance with some embodiments.

FIG. 7A illustrates a needle shaft of the introducer needle with a needle slot extending along at least a portion of a top of the needle shaft in accordance with some embodiments.

FIG. 7B illustrates the needle shaft with a needle-slot opening in a proximal portion of the needle shaft in accordance with some embodiments.

FIG. 7C illustrates the needle shaft with a needle-slot opening in a distal portion of the needle shaft in accordance with some embodiments.

FIG. 7D illustrates the needle shaft with a needle slot extending along at least a portion of a side of the needle shaft in accordance with some embodiments.

FIG. 7E illustrates the needle shaft with a partially helical needle slot extending along the needle shaft in accordance with some embodiments.

FIG. 7F illustrates the needle shaft with another partially helical needle slot extending along the needle shaft in accordance with some embodiments.

FIG. 8A illustrates a detailed view of a distal portion of the needle shaft in which the needle slot bisects a heel of a bevel of a needle tip accordance with some embodiments.

FIG. 8B illustrates a detailed view of the distal portion of the needle shaft in which the needle slot intersects both the heel and a tip bevel of the bevel without bisecting the heel or tip bevel of the bevel in accordance with some embodiments.

FIG. 9A illustrates a transverse cross section of the needle shaft in which opposite needle-slot walls face each other and are parallel to each other in accordance with some embodiments.

FIG. 9B illustrates a transverse cross section of the needle shaft in which the opposite needle-slot walls face each other and are oblique to each other in accordance with some embodiments.

FIG. 9C illustrates a transverse cross section of the needle shaft in which the opposite needle-slot walls face away from a bottom of the needle slot in accordance with some embodiments.

FIG. 9D illustrates a transverse cross section of the needle shaft in which the opposite needle-slot walls face away from the bottom of the needle slot in accordance with some embodiments.

FIG. 9E illustrates a transverse cross section of the needle shaft in which the opposite needle-shaft walls are parallel in accordance with some embodiments.

FIG. 10 illustrates a RICC of the RICC insertion assembly in accordance with some embodiments.

FIG. 11 illustrates a detailed view of a distal portion of a catheter tube of the RICC in accordance with some embodiments.

FIG. 12 illustrates a transverse cross section of the distal portion of the catheter tube in accordance with some embodiments.

FIG. 13 illustrates another transverse cross section of the distal portion of the catheter tube in accordance with some embodiments.

FIG. 14 illustrates a longitudinal cross section of the distal portion of the catheter tube in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

As set forth above with respect to the Seldinger technique, the number of steps are time consuming, handling the number of medical devices is awkward, and both of the foregoing can lead to patient trauma. In addition, there is a relatively high potential for touch contamination due to the number of medical devices that need to be interchanged during the Seldinger technique. As such, there is a need to reduce the number of steps and medical devices involved in introducing a catheter such as a CVC into a patient and advancing the catheter through a vasculature thereof.

Disclosed herein are introduce needles for insertion assemblies of RICCs and methods that address the foregoing. For example, an introducer needle can include a needle shaft, a sheath over the needle shaft, and a needle hub around a proximal portion of each of the needle shaft and the sheath. The needle shaft includes a needle slot extending from the proximal portion of the needle shaft through a distal needle tip, which needle tip includes a bevel having a tip bevel and a heel. The needle shaft includes opposite needle-slot walls that either a) face each other and are parallel or oblique to each other or b) face toward or away from a bottom of the needle slot. Additionally or alternatively, opposite needle-shaft walls can be parallel to each other. The sheath over the needle shaft seals the needle slot thereunder but for a sheath opening in a proximal portion of the sheath.

The foregoing features as well as other features of the introducer needles, the RICC insertion assemblies, and methods disclosed herein will become more apparent to those of skill in the art in view of the accompanying drawings and following description, which describe particular embodiments of the introducer needles, the RICC insertion assemblies, and methods in greater detail beginning with the RICC insertion assemblies. However, it should be understood the RICCs of the RICC insertion assemblies are but one type of catheter that can be incorporated into catheter insertion assemblies like those disclosed herein. Indeed, peripherally inserted central catheters (“PICCs”), dialysis catheters, or the like can also be incorporated into catheter insertion assemblies and methods like those disclosed herein.

### RICC Insertion Assemblies

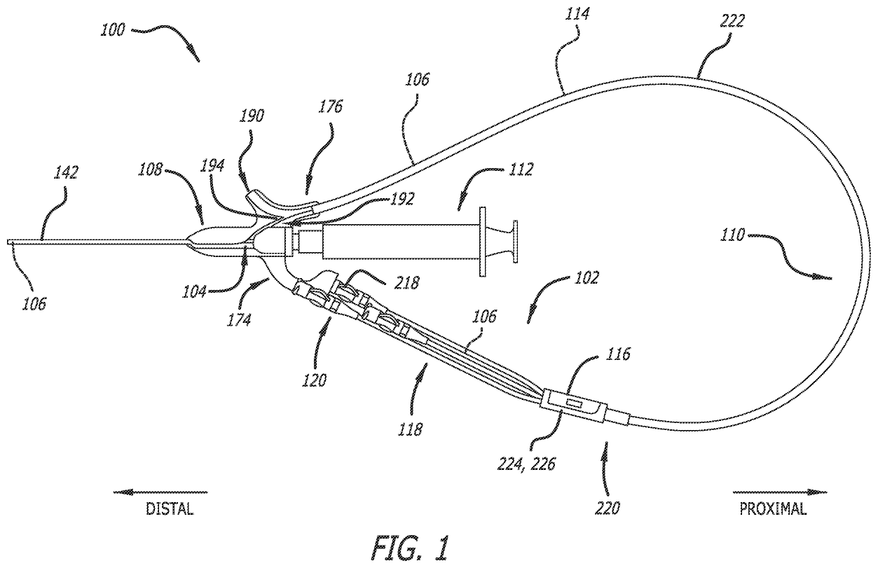

FIG. 1 illustrates a RICC insertion assembly 100 in accordance with some embodiments.

As shown, the RICC insertion assembly 100 includes a RICC 102, an introducer needle 104, an access guidewire 106, and a coupler 108 coupling the RICC 102, the introducer needle 104, and the access guidewire 106 together in a ready-to-operate state of the RICC insertion assembly 100. As set forth in more detail below, the proximal end of the access guidewire 106 is coupled to the coupler 108 and the distal end of the access guidewire 106 is disposed in the needle lumen 158 of the introducer needle 104 in the ready-to-operate state of the RICC insertion assembly 100. This enforces a loop 110 in the access guidewire 106, which loop 110 the RICC 102 is disposed over in the ready-to-operate state of the RICC insertion assembly 100 keeping the RICC insertion assembly 100 in a relatively compact form.

The RICC insertion assembly 100 can further include a syringe 112 fluidly coupled to the introducer needle 104 in the ready-to-operate state of the RICC insertion assembly 100. As set forth below, the splittable sealing module 196 seals around the proximal portion of the introducer needle 104 and the distal portion of the access guidewire 106 when the sealing-module insert 198 is compressed in the sealing-module cavity 178 in one or more states of the RICC insertion assembly 100. In particular, the splittable sealing module 196 seals over the sheath opening 162 of the sheath 142 that opens to the needle slot 150 of the needle shaft 144. Outside of the splittable sealing module 196, the sheath 142 seals the needle slot 150 of the needle shaft 144. Such seals enable the syringe 112 to aspirate blood in accordance with the blood-aspirating step of the method set forth below.

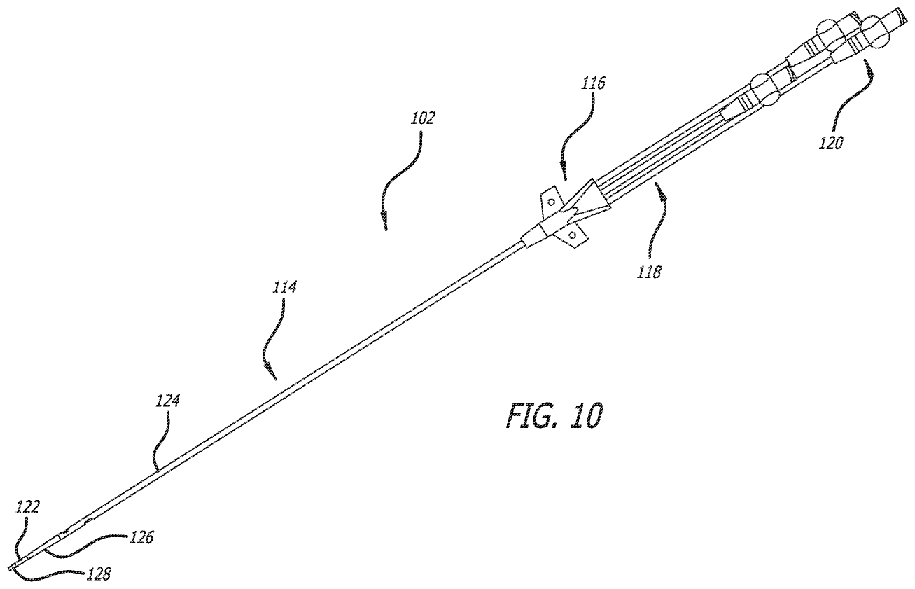

FIG. 10 illustrates the RICC 102 of the RICC insertion assembly 100 in accordance with some embodiments.

As shown, the RICC 102 includes a catheter tube 114, a catheter hub 116, one or more extension legs 118, and one or more extension-leg connectors 120.

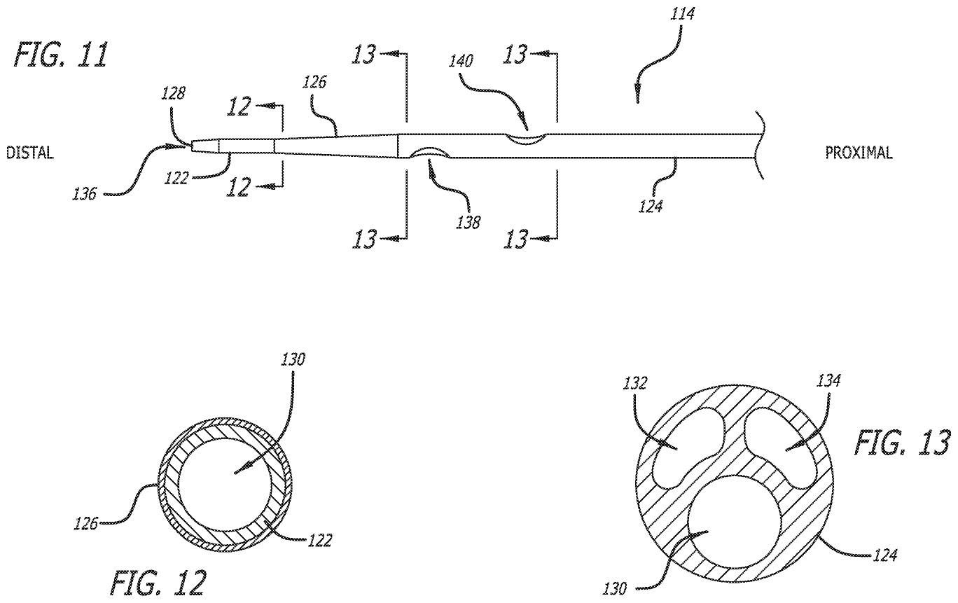

FIGS. 11-14 illustrate various views of the catheter tube 114 of the RICC 102 in accordance with some embodiments.

The catheter tube 114 includes a first section 122 in a distal portion of the catheter tube 114, a second section 124 in the distal portion of the catheter tube 114 proximal of the first section 122, and a tapered junction 126 between the first and second sections 122 and 124 of the catheter tube 114.

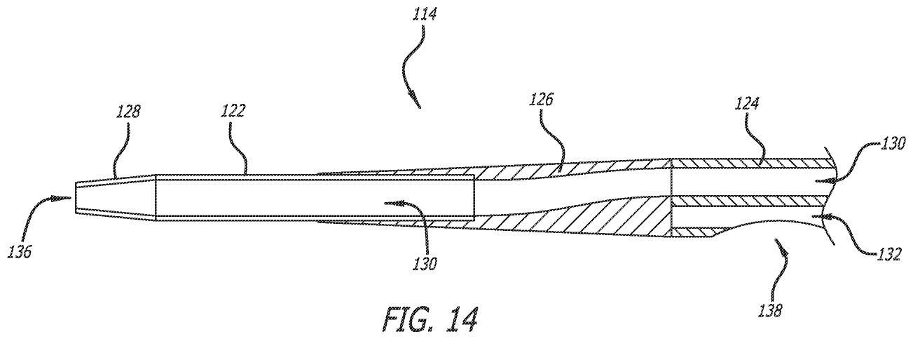

The first section 122 of the catheter tube 114 includes a catheter tip 128 having a relatively short taper from an outer diameter of a distal portion of the first section 122 distal of the junction 126 to an outer diameter of a distal end of the first section 122. The taper of the catheter tip 128 is configured for immediate dilation of tissue about a needle tract established with the introducer needle 104 up to the outer diameter of the distal portion of the first section 122 of the catheter tube 114. As best shown in FIG. 14, the first section 122 of the catheter tube 114 also includes a proximal portion disposed in a bore of a distal portion of the junction 126 and fixedly coupled thereto such as by a solvent bond, an adhesive bond, or a heat weld.

The second section 124 of the catheter tube 114 includes a consistent outer diameter over its length from a distal end of the second section 124 to a proximal end of the second section 124. The consistent diameter of the second section 124 of the catheter tube 114 is configured for smooth insertion into the needle tract and targeted vasculature subsequent to any dilation by the first section 122 of the catheter tube 114 and the junction 126. The distal end of the second section 124 of the catheter tube 114 has a flat face flush with the flat-faced proximal end of the junction 126 and fixedly coupled thereto such as by a solvent bond, an adhesive bond, or a heat weld.

The junction 126 includes a taper over its length from a proximal end of the junction 126 to a distal end of the junction 126. The taper of the junction 126 is configured for immediate dilation of the tissue about the needle tract from the outer diameter of the proximal portion of the first section 122 of the catheter tube 114 to the outer diameter of the second section 124 of the catheter tube 114. An abluminal surface of the junction 126 smoothly transitions from an abluminal surface of the first section 122 of the catheter tube 114 to an abluminal surface of the second section 124 of the catheter tube 114 without edges that catch on skin when the catheter tube 114 is inserted into the needle tract. In addition to the edges being minimal to negligible, the edges can include solvent-interdiffused polymeric material of the polymeric materials from which the catheter tube 114 is formed, which smoothens the transitions from the first section 122 of the catheter tube 114 to the junction 126 and from the junction 126 to the second section 124 of the catheter tube 114. Notably, the junction 126 has a length approximately commensurate with a length of an exposed portion of the first section 122 of the catheter tube 114 or between lengths of exposed portions of the first and second sections 122 and 124 of the catheter tube 114. As such, the length of the exposed portion of the first section 122 of the catheter tube 114 is less than the length of the junction 126 up to approximately commensurate with the length of the junction 126.

The first section 122 of the catheter tube 114 is formed of a first polymeric material (e.g., a polytetrafluoroethylene, a polypropylene, or a polyurethane) having a first durometer. The second section 124 of the catheter tube 114 is formed of a second polymeric material (e.g., a polyvinyl chloride, a polyethylene, another polyurethane, or a silicone) having a second durometer less than the first durometer. For example, the first section 122 of the catheter tube 114 can be formed of a first polyurethane having the first durometer while the second section 124 of the catheter tube 114 can be formed of a second, different polyurethane (e.g., a same or different diisocyanate or triisocyanate reacted with a different diol or triol, a different diisocyanate or triisocyanate reacted with a same or different diol or triol, a same diisocyanate or triisocyanate reacted with a same diol or triol under different conditions or with different additives, etc.) having the second durometer less than the first durometer. Indeed, polyurethanes are advantageous for the catheter tube 114 in that polyurethanes can be relatively rigid at room-temperature but become more flexible in vivo at body temperature, which reduces irritation to vessel walls as well as phlebitis. Polyurethanes are also advantageous in that they can be less thrombogenic than some other polymers. The junction 126 is formed of the second polymeric material or a third polymeric material (e.g., yet another polyurethane) having a third durometer less than the first durometer and greater than, approximately equal to, or less than the second durometer.

It should be understood the first durometer of the first polymeric material, the second durometer of the second polymeric material, and the third durometer of the third polymeric material can be on different scales (e.g., Type A or Type D). With this understanding, the second durometer of the second polymeric material or the third durometer of the third polymeric material might not be numerically less than the first durometer of the first polymeric material when the second durometer or the third durometer is less than the first durometer. Indeed, the hardness of the second polymeric material or the third polymeric material can still be less than the hardness of the first polymeric material as the different scales—each of which ranges from 0 to 100—are designed for characterizing different materials in groups of the materials having a like hardness.

In accordance with the first section 122 of the catheter tube 114, the second section 124 of the catheter tube 114, and the junction 126 between the first and second sections 122 and 124 of the catheter tube 114 set forth above, the catheter tube 114 possesses a column strength sufficient to prevent buckling of the catheter tube 114 when inserted into a needle tract established by with the introducer needle 104. The column strength of the catheter tube 114 is also sufficient to prevent buckling of the catheter tube 114 when advanced through a vasculature of a patient without dilation of tissue about the needle tract or any blood vessels of the vasculature beforehand with a separate dilator.

The catheter tube 114 includes one or more catheter-tube lumens extending through the catheter tube 114; however, only one catheter-tube lumen typically extends from a proximal end of the catheter tube 114 to a distal end of the catheter tube 114 in a multiluminal RICC (e.g., a diluminal RICC, a triluminal RICC, a tetraluminal RICC, a pentaluminal RICC, a hexaluminal RICC, etc.). (See FIGS. 11-14.) Indeed, the first section 122 of the catheter tube 114 typically includes a single lumen therethrough as shown in FIGS. 12 and 14.

The catheter hub 116 is coupled to a proximal portion of the catheter tube 114. The catheter hub 116 includes one or more catheter-hub lumens corresponding in number to the one-or-more catheter-tube lumens. The one-or-more catheter-hub lumens extends through an entirety of the catheter hub 116 from a proximal end of the catheter hub 116 to a distal end of the catheter hub 116.

Each extension leg of the one-or-more extension legs 118 is coupled to the catheter hub 116 by a distal portion thereof. The one-or-more extension legs 118 respectively include one or more extension-leg lumens, which, in turn, correspond in number to the one-or-more catheter-hub lumens. Each extension-leg lumen of the one-or-more extension-leg lumens extends through an entirety of the extension leg from a proximal end of the extension leg to a distal end of the extension leg.

Each extension-leg connector of the one-or-more extension-leg connectors 120 is over a proximal portion of an extension leg of the one-or-more extension legs 118. For example, each extension-leg connector of the one-or-more extension-leg connectors 120 can be a Luer connector over a proximal portion of an extension leg of the one-or-more extension legs 118. Through such an extension-leg connector, a corresponding extension leg and the extension-leg lumen thereof can be connected to another medical device and a lumen thereof. However, in the ready-to-operate state of the RICC insertion assembly 100 at least one extension-leg connector (e.g., the extension-leg connector including part of the primary lumen 130 of the RICC 102) is indirectly connected via the intervening access-guidewire hub 218 to the access guidewire-connecting side arm 174 of the coupler 108 to enforce the loop 110 in the access guidewire 106 and the RICC 102 thereover.

As shown, the RICC 102 is a triluminal RICC including a set of three lumens; however, the RICC 102 is not limited to the set of the three lumens as set forth above. The set of three lumens includes a primary lumen 130, a secondary lumen 132, and a tertiary lumen 134 formed of fluidly connected portions of three catheter-tube lumens, three catheter-hub lumens, and three extension-leg lumens. The primary lumen 130 has a primary-lumen aperture 136 in the distal end of the first section 122 of the catheter tube 114, which corresponds to the distal end of the catheter tube 114 and a distal end of the RICC 102. The secondary lumen 132 has a secondary-lumen aperture 138 in a side of the distal portion of the catheter tube 114. The tertiary lumen 134 has a tertiary-lumen aperture 140 in the side of the distal portion of the catheter tube 114 proximal of the secondary-lumen aperture 138.

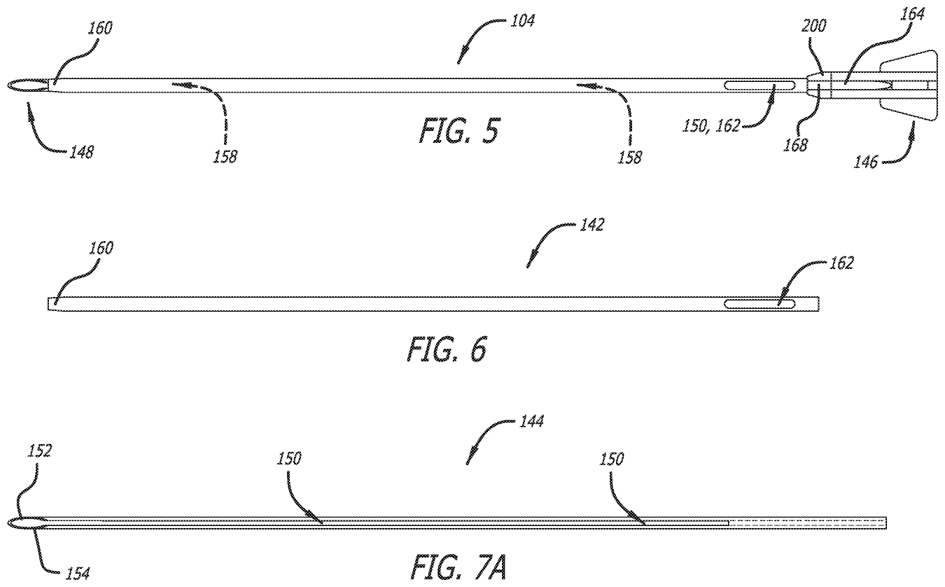

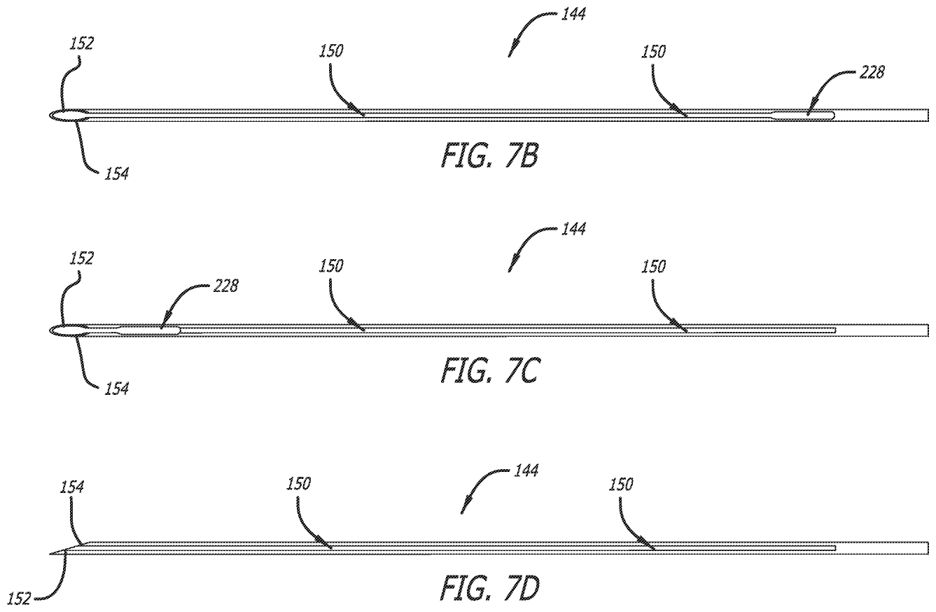

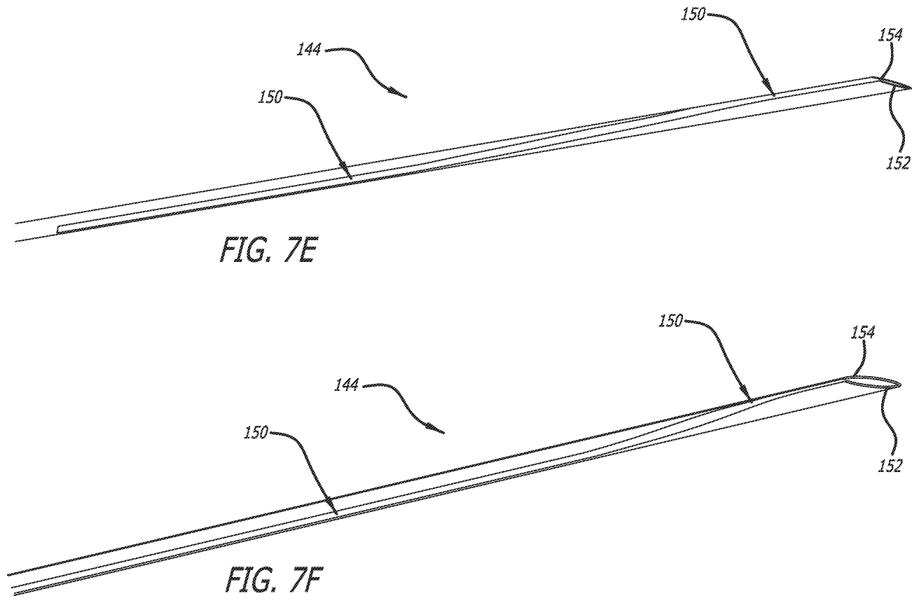

FIG. 5 illustrates a top view of the introducer needle 104 of the RICC insertion assembly 100 in accordance with some embodiments. FIG. 6 illustrates a sheath 142 of the introducer needle 104 in accordance with some embodiments. FIGS. 7A-7F, 8A, 8B, and 9A-9E illustrate various views of a needle shaft 144 of the introducer needle 104 in accordance with some embodiments.

As shown, the introducer needle 104 includes the needle shaft 144, the sheath 142 over the needle shaft 144, and a needle hub 146 in a proximal portion of the introducer needle 104 over both a proximal-end portion of the needle shaft 144 and a proximal-end portion of the sheath 142. In addition, the introducer needle 104 can be considered to include the proximal portion 200 of the sealing-module insert 198 coupled to the distal portion of the needle hub 146, particularly when the proximal portion 200 of the sealing-module insert 198 is fixedly, as opposed to removably, coupled to the needle hub 146. In at least the ready-to-operate state of the RICC insertion assembly 100, the needle shaft 144 and the sheath 142 extend from the needle hub 146, through the splittable sealing module 196, and out a distal end of the coupler housing 172.

The needle shaft 144 includes a needle tip 148 in a distal portion of the needle shaft 144 and a longitudinal needle slot 150 extending from the proximal portion of the needle shaft 144 through the needle tip 148.

The needle tip 148 includes a bevel having a tip or secondary bevel 152 and a primary bevel 154 proximal of the tip bevel 152, wherein the primary bevel 154 terminates in a proximal heel. A tip-bevel angle of the tip bevel 152 is greater than a primary-bevel angle (e.g., 7°-21°, such as 14°) of the primary bevel 154 such that the bevel provides a smooth transition over the needle tip 148. Such a needle tip is thusly configured for establishing a needle tract from an area of skin into a blood-vessel lumen of a patient in accordance with the needle tract-establishing step of the method set forth below. Notably, a top of the needle shaft 144 includes the heel of the bevel, a bottom of the needle shaft 144 includes the tip bevel 152 of the bevel, and a side of the needle shaft 144 is between the heel and tip bevel 152 of the bevel.

The needle slot 150 extends from the proximal portion of the needle shaft 144 through the needle tip 148, thereby forming a needle channel 156 along a majority of a length (e.g., about 3-5 inches, such as about 3.4 inches or 4.3 inches) of the needle shaft 144 as opposed to a needle lumen therethrough.

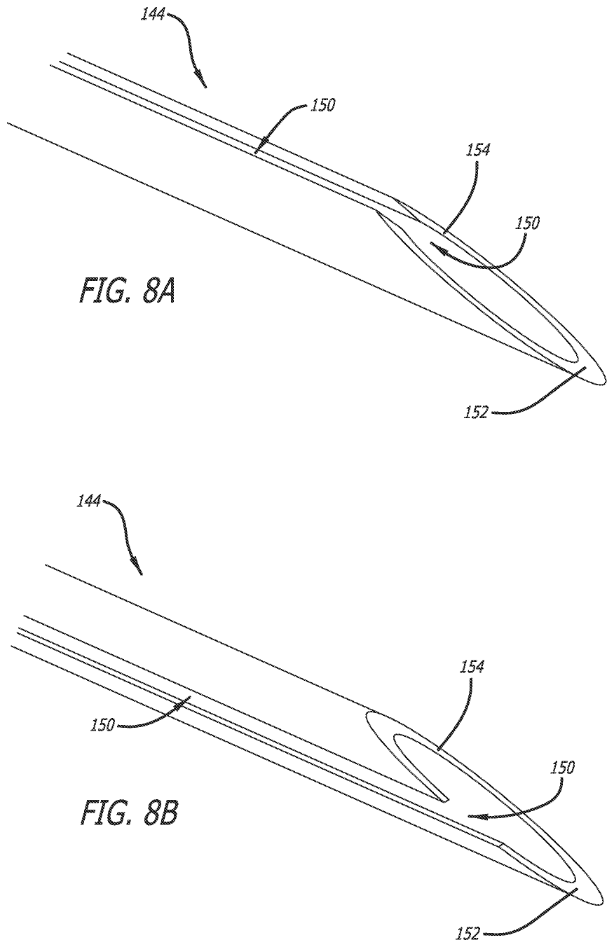

The needle slot 150 can extend along at least a portion of the top of the needle shaft 144 such as a minority of the top of the needle shaft 144 up to a majority of the top of the needle shaft 144 but short (e.g., by about 0.2-0.3 inches) of a proximal end of the needle shaft 144. The needle slot 150 can even extend along an entirety of the top of the needle shaft 144 including the proximal end of the needle shaft 144 as alluded to in FIG. 7A. As shown in FIGS. 7A-7C, for example, the needle slot 150 is linear and extends along the majority of the top of the needle shaft 144 from the proximal portion of the needle shaft 144 through the needle tip 148, short of the proximal end of the needle shaft 144 and bisecting the heel of the bevel as shown in FIG. 8A. However, the needle slot 150 can be non-linear, too, such as at least partially helical as set forth below.

The needle slot 150 can extend along at least a portion of the bottom of the needle shaft 144 such as a minority of the bottom of the needle shaft 144 up to a majority of the bottom of the needle shaft 144 but short (e.g., by about 0.2-0.3 inches) of the proximal end of the needle shaft 144. The needle slot 150 can even extend along an entirety of the bottom of the needle shaft 144 including the proximal end of the needle shaft 144. While not shown, the needle slot 150 can be linear and extend along the majority of the bottom of the needle shaft 144 from the proximal portion of the needle shaft 144 through the needle tip 148, short of the proximal end of the needle shaft 144 and bisecting the tip bevel 152 of the bevel. However, the needle slot 150 can be non-linear, too, such as at least partially helical as set forth below.

The needle slot 150 can extend along at least a portion of the side of the needle shaft 144 such as a minority of the side of the needle shaft 144 up to a majority of the side of the needle shaft 144 but short (e.g., by about 0.2-0.3 inches) of the proximal end of the needle shaft 144. The needle slot 150 can even extend along an entirety of the side of the needle shaft 144 including the proximal end of the needle shaft 144. As shown in FIG. 7D, for example, the needle slot 150 can be linear and extend along the majority of the side of the needle shaft 144 from the proximal portion of the needle shaft 144 through the needle tip 148, short of the proximal end of the needle shaft 144 and intersecting the heel, the tip bevel 152, or both the heel and tip bevel 152 of the bevel without bisecting the heel or tip bevel 152 of the bevel as shown in FIG. 8B. However, the needle slot 150 can be non-linear, too, such as at least partially helical as set forth below.

The needle slot 150 can, in view of the foregoing, extend along at least a portion of the top, bottom, or side of the needle shaft 144 such as a minority of the top, bottom, or side of the needle shaft 144 up to a majority of the top, bottom, or side of the needle shaft 144 but short of the proximal end of the needle shaft 144. And such a needle slot is not limited to being linear. Indeed, the needle slot 150 can be non-linear such as at least partially helical as set forth below. By varying the implementation of the needle slot 150, a flexural strength of the needle shaft 144 can be varied as desired. For example, when the needle slot 150 bisects the heel of the bevel as shown in at least FIGS. 7A-7C, 7E, 7F, and 8A, the flexural strength of the needle shaft 144 is greater along the top and bottom of the needle shaft 144 about at least the distal portion of the needle shaft 144 when compared to the needle slot 150 in the side of the needle shaft 144. This can be important because the introducer needle 104 is typically flexed into the top or bottom of the needle shaft 144 about the distal portion of the needle shaft 144 during a venipuncture. Also, by varying the implementation of the needle slot 150 in combination with the coupler-housing slot 182 of the coupler housing 172, the access guidewire 106 can be easily inserted into the needle shaft 144 during assembly of the RICC insertion assembly 100 and removed from the coupler-housing slot 182 during the introducer needle-withdrawing step of the method set forth below or subsequent thereto such as during the access guidewire-withdrawing step.

As shown in FIGS. 7E and 7F, for example, the needle slot 150 can form at least part of a helix (e.g., a quarter helix, a half helix, a three-quarter helix, etc.) between the proximal portion of the needle shaft 144 through the needle tip 148, short (e.g., by about 0.2-0.3 inches) of the proximal end of the needle shaft 144 and bisecting the heel of the bevel. The needle slot 150 configured as such a helix allows the access guidewire 106 to be easily inserted in the side of the needle shaft 144 during assembly of the RICC insertion assembly 100 and removed from the top or bottom of the needle shaft 144 during the introducer needle-withdrawing step of the method set forth below or subsequent thereto such as during the access guidewire-withdrawing step. Notably, the needle slot 150 of FIG. 7E exemplifies a more gradual quarter helix than that of FIG. 7F, the needle slot 150 of FIG. 7E turning more gradually from the side of the needle shaft 144 in a medial portion of the needle shaft 144 to the top of the needle shaft 144 where it bisects the heel of the bevel. The needle slot 150 of FIG. 7F turns more rapidly from the side of the needle shaft 144 in the distal portion thereof to the top of the needle shaft 144 where it bisects the heel of the bevel. While the needle slot 150 of FIG. 7E and that of FIG. 7F turn from the side of the needle shaft 144 to the top of the needle shaft 144, the needle slot 150 can alternatively turn from the side of the needle shaft 144 to the bottom of the needle shaft 144 such that it bisects the tip bevel 152 of the bevel. The needle slot 150 can alternatively turn from the top or bottom of the needle shaft 144 to the side of the needle shaft 144 such that it intersects the heel, the tip bevel 152, or both the heel and the tip bevel 152 of the bevel without bisecting the heel or tip bevel 152 of the bevel. Furthermore, the helix can have a handedness selected from a right-handed helix and a left-handed-helix for handed RICC insertion assemblies.

The needle slot 150 has a needle-slot width sized in accordance with at least an outer diameter of the access guidewire 106, which allows the access guidewire 106 to pass from the proximal portion of the needle shaft 144 through the needle tip 148 when the introducer needle-withdrawing step of the method set forth below is performed. Indeed, the needle slot 150 can have a constant needle-slot width sized in accordance with at least an outer diameter of the access guidewire 106 such as slightly larger than an outer diameter of the access guidewire 106. For example, the slot width can range from about 0.018 inches to about 0.035 inches (e.g., 0.02 inches). However, the needle slot 150 can alternatively have a non-constant needle-slot width sized in accordance with at least an outer diameter of the access guidewire 106. For example, the needle slot 150 can include a taper, a reverse taper, or one or more stepwise changes in the needle-slot width from the proximal portion of the needle shaft 144 through the needle tip 148. When the needle slot 150 is tapered or includes the one-or-more stepwise changes in a same direction as the taper, the slot width can vary from the proximal portion of the needle shaft 144 to the distal portion of the needle shaft 144 through the needle tip 148. For example, the slot width can vary from about 0.025 inches in the proximal portion of the needle shaft 144 to about 0.018 inches in the distal portion of the needle shaft 144 through the needle tip 148. Advantageously, such a slot width facilitates insertion of the access guidewire 106 in the needle slot 150 during assembly of the RICC insertion assembly 100. In addition, such a slot width guides the blade 212 of the splittable sealing module 196 in cutting the sheath 142 away from the needle shaft 144 in the introducer needle-withdrawing step of the method set forth below. Alternatively or additionally, the needle slot 150 can include one or more needle-slot openings 228 wider (e.g., about 0.020 inches for a 0.018-inch needle-slot width) than a remainder of the needle slot 150. In an example, the needle slot 150 can include a distal needle-slot opening 228, which can interact with a safety mechanism sealed around the distal needle-slot opening 228 for preventing at least proximal movement of the introducer needle 104 until desired such as during the introducer needle-withdrawing step of the method set forth below. In another example, the needle slot 150 can include a proximal needle-slot opening 228, which facilitates insertion of the access guidewire 106 in the needle slot 150 during assembly of the RICC insertion assembly 100.

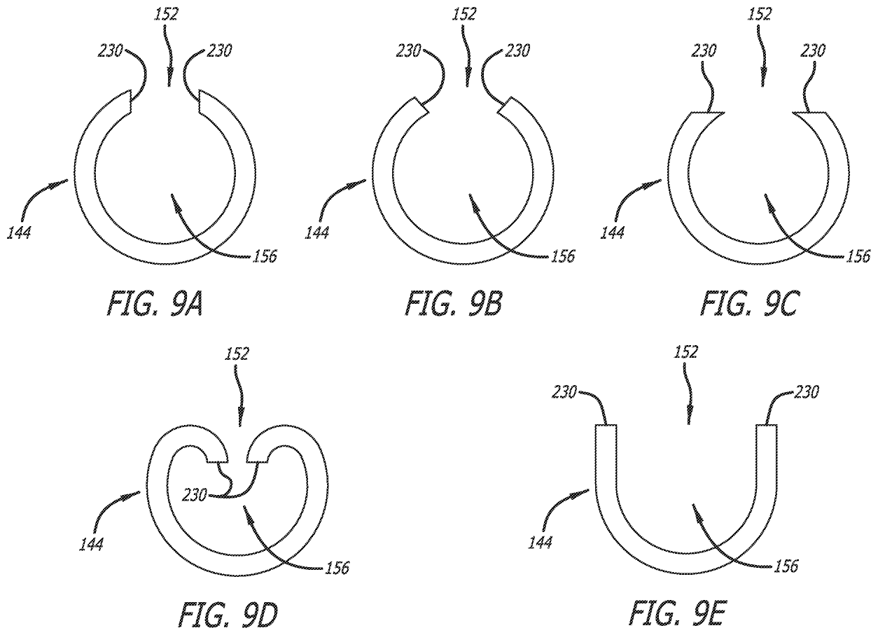

As shown in FIG. 9A, opposite needle-slot walls 230 can face each other and be parallel to each other, which is consistent with the needle slot 150 being cut into the needle shaft 144 such as by a laser or machining (e.g., electrical discharge machining). As shown in FIG. 9B, the opposite needle-slot walls 230 can face each other but be oblique to each other, which is consistent with the needle shaft 144 being stamped or rolled to form the needle slot 150 such as by a milling machine. As shown in FIG. 9C, the opposite needle-slot walls 230 can face away from a bottom of the needle slot 150, which is consistent with the needle slot 150 being ground into the needle shaft 144. As shown in FIG. 9D, the opposite needle-slot walls 230 can face toward the bottom of the needle slot 150, which is consistent with the opposite needle-slot walls 230 being bent in toward the bottom of the needle slot 150. Advantageously, with the opposite needle-slot walls 230 bent in toward the bottom of the needle slot 150, the flexural strength of the needle shaft 144 is increased. As shown in FIG. 9E, opposite needle-shaft walls 230 can be parallel to each other for a ‘U’ shape, which is consistent with the needle shaft 144 being stamped or rolled to form the needle slot 150. Notably, edges of the needle-slot walls 230 can be finished (e.g., ground, polished, etc.). The edges of the needle-slot walls 230 being finished minimizes or eliminates sharp, access guidewire-fraying edges of the needle slot 150.

Notably, the needle shaft 144 includes the needle channel 156, whereas the introducer needle 104 includes a needle lumen 158. This is because the needle lumen 158 results from the combination of the needle shaft 144 and the sheath 142 over the needle shaft 144. Indeed, the sheath 142 over the needle shaft 144 seals the needle channel 156 forming the needle lumen 158 of the introducer needle 104 and enabling the syringe 112 to aspirate blood in accordance with the blood-aspirating step of the method set forth below.

The sheath 142 includes a sheath tip 160 in a distal portion of the sheath 142 and a sheath opening 162 in a side of the proximal portion of the sheath 142.

The sheath tip 160 includes a relatively short taper from an outer diameter of the distal portion of the sheath 142 to an outer diameter of a distal end of the sheath 142, the latter of which is commensurate with an outer diameter of the distal portion of the needle shaft 144. The taper has a taper angle less than the primary-bevel angle of the primary bevel 154 of the needle tip 148, which, in turn, is less than the tip-bevel angle of the tip bevel 152 of the needle tip 148. The sheath tip 160 including such a taper is configured to provide a smooth transition from the needle tip 148 to the sheath body for the needle tract-establishing step of the method set forth below.

The sheath opening 162 opens to the needle slot 150 of the needle shaft 144 allowing the access guidewire 106 to pass through the sheath opening 162 and into the needle slot 150 in the ready-to-operate state of the RICC insertion assembly 100. Thus, the sheath opening 162 has a width approximately commensurate with a width of the needle slot 150 thereunder, which needle slot 150, in turn, is sized in accordance with the diameter of the access guidewire 106. The sheath opening 162 also has a length sufficient to allow the access guidewire 106 to pass through the sheath opening 162 and into the needle slot 150 while also accommodating the blade 212 of the splittable sealing module 196 under a distal end of the sheath opening 162. Notably, the sheath 142 over the needle shaft 144 seals the needle slot 150 thereunder except for that under the sheath opening 162. However, the splittable sealing module 196 seals over the needle slot 150 exposed by the sheath opening 162 by sealing the proximal portions of the needle shaft 144 and the sheath 142 therein, thereby enabling the syringe 112 to aspirate blood in accordance with the blood-aspirating step of the method set forth below.

The sheath 142 can be a cuttable or a splittable sheath configured for respectively cutting or splitting the sheath 142 away from the needle shaft 144 to allow the access guidewire 106 to escape from the needle shaft 144 by way of the needle slot 150. When configured to be cut away from the needle shaft 144, the sheath 142 can be formed of a polymeric material such as polyurethane that facilitates the cutting of the sheath 142 away from the needle shaft 144. When configured to be split away from the needle shaft 144, the sheath 142 can include one or more weakened portions (e.g., a longitudinal pattern of perforations, longitudinal grooves, etc.) of the sheath 142 that facilitate the splitting of the sheath 142 away from the needle shaft 144.

The sheath 142, or a sheath body thereof, is formed of a polymeric material configured to facilitate a smooth, consistent insertion of the introducer needle 104 from an area of skin to a blood-vessel lumen of a patient in accordance with the needle tract-establishing step of the method set forth below. In addition, the polymeric material has mechanical properties at a thickness of the sheath 142 sufficient to withstand collapse of the sheath 142 into the needle slot 150 of the needle shaft 144 when the blood-aspirating step of the method set forth below is performed, notably, while also facilitating the cutting or splitting of the sheath 142 off the needle shaft 144 in accordance with at least the introducer needle-withdrawing step of the method set forth below for the cutting of the sheath 142 off the needle shaft 144. Such a polymeric material can include, but is not limited to, polyethylene, polypropylene, polyurethane, or polytetrafluoroethylene (“PTFE”). In an example, the sheath 142 can be polyurethane in embodiments of the RICC insertion assembly 100 in which the sheath 142 is cut away from the needle shaft 144 by the blade 212. In another example, the sheath 142 can be PTFE or even expanded PTFE (“ePTFE”) in embodiments of the RICC insertion assembly 100 in which the sheath 142 is split away from the needle shaft 144. When the sheath 142 is, for example, ePTFE, the sheath 142 need not include the one-or-more weakened portions of the sheath 142 for the splitting of the sheath 142 away from the needle shaft 144 because ePTFE, itself, facilitates the splitting of the sheath 142 away from the needle shaft 144 on account of the longitudinal arrangement of polymer chains in the ePTFE.

The needle hub 146 includes an access-guidewire channel 164 in a distal portion of the needle hub 146 and a needle-hub connector 166 in a proximal portion of the needle hub 146.

The access-guidewire channel 164 of the needle hub 146 is configured to allow the access guidewire 106 to pass over the needle hub 146 and direct the access guidewire 106 into the access-guidewire passageway 206 of the splittable sealing module 196. The access-guidewire channel 164 is open such that the access guidewire 106 lies in the access-guidewire channel 164 in at least the ready-to-operate state of the RICC insertion assembly 100. Advantageously, the open access-guidewire channel 164 allows the access guidewire 106 to remain in place when the introducer needle 104 is withdrawn from the RICC insertion assembly 100 in accordance with the introducer needle-withdrawing step of the method set forth below.

Notably, the access-guidewire channel 164 of the needle hub 146 transitions into an access-guidewire channel 168 of the proximal portion 200 of the sealing-module insert 198 coupled to the distal portion of the needle hub 146. When the proximal portion 200 of the sealing-module insert 198 is combined with the distal portion 202 of the sealing-module insert 198, the access-guidewire channel 168 of the proximal portion 200 of the sealing-module insert 198 combines with that of the distal portion 202 of the sealing-module insert 198 to form the access-guidewire passageway 206 of the splittable sealing module 196. The needle hub 146 including the proximal portion 200 of the sealing-module insert 198 coupled thereto is configured to be removably disposed in the needle-hub receptacle 180 and the sealing-module cavity 178 of the coupler housing 172, respectively. As set forth below, the distal portion of the needle hub 146 axially compresses the proximal portion 200 of the sealing-module insert 198 in the sealing-module cavity 178 when the needle hub 146 is disposed in the needle-hub receptacle 180 and the proximal portion 200 of the sealing-module insert 198 is disposed in the sealing-module cavity 178. Axial compression of the proximal portion 200 of the sealing-module insert 198 in the sealing-module cavity 178, in turn, radially compresses both the proximal and distal portions 200 and 202 of the sealing-module insert 198 in the sealing-module cavity 178.

The needle-hub connector 166 includes a needle-hub bore 170 and an optional needle-hub flange (not shown) about the needle-hub connector 166.

The needle-hub bore 170 of the needle-hub connector 166 is configured to accept a syringe tip of the syringe 112 therein for fluidly connecting the introducer needle 104 to the syringe 112. Indeed, the needle-hub bore 170 can have a Luer taper (e.g., a 6% taper) configured to accept the syringe tip therein, which syringe tip can be complementarily configured with a Luer taper.

The needle-hub flange of the needle-hub connector 166 is configured to screw together with internal threads of a threaded collar around the syringe tip of the syringe 112. While the threaded collar of the syringe 112 is optional, the needle-hub flange advantageously provides a so-called Luer lock-style connection with the internal threads of the threaded collar when both are present. This provides added security against inadvertent disconnection of the introducer needle 104 and the syringe 112 over that provided by an otherwise Luer slip-style connection.

FIGS. 1-3 illustrate various views of the coupler 108 of the RICC insertion assembly 100 in accordance with some embodiments.

As shown, the coupler 108 includes a coupler housing 172, an access guidewire-connecting side arm 174, and, optionally, a splittable casing-holding side arm 176 when the RICC insertion assembly 100 also includes the keeper 220.

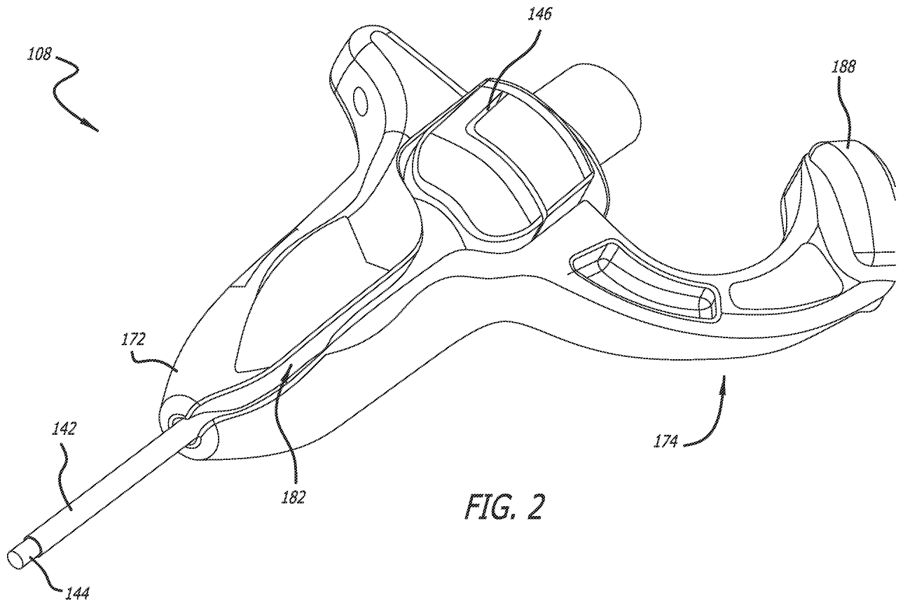

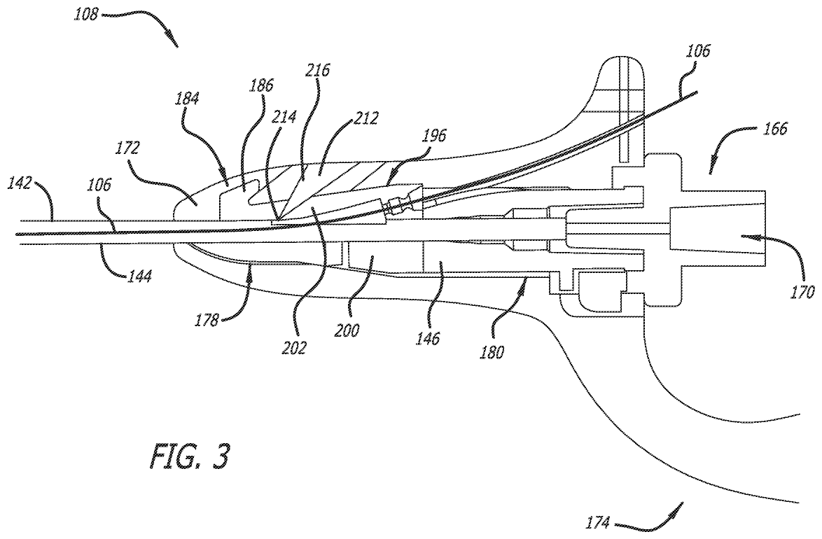

The coupler housing 172 includes a sealing-module cavity 178, a needle-hub receptacle 180 proximal of the sealing-module cavity 178, and a longitudinal coupler-housing slot 182 formed along a length of the coupler housing 172. (See FIG. 3, which includes the proximal and distal portions 200 and 202 of the sealing-module insert 198 disposed in the sealing-module cavity 178. FIG. 3 also includes the needle hub 146 of the introducer needle 104 disposed in the needle-hub receptacle 180.) Notably, the coupler housing 172 can be formed into a bullet-shaped body configured to be comfortably held underhand (e.g., cradled) or overhand in either a left hand for a left-handed venipuncture or a right hand for a right-handed venipuncture with the RICC insertion assembly 100. To further facilitate such venipunctures, an exterior of the coupler housing 172 can be textured with grip-enhancing ridges (e.g., transverse or circumferential ridges), protrusions, or the like.

The sealing-module cavity 178 is configured to hold the proximal and distal portions 200 and 202 of the sealing-module insert 198 therein. Indeed, the sealing-module cavity 178 includes at least the distal portion 202 of the sealing-module insert 198 captively disposed therein in each state of the one-or-more states of the RICC insertion assembly 100. For example, the sealing-module cavity 178 can include a catch 184 in a distal portion thereof, wherein the catch 184 is configured to accept therein a protrusion 186 of the distal portion 202 of the sealing-module insert 198 for captive disposal of the distal portion 202 of the sealing-module insert 198 in the sealing-module cavity. The sealing-module cavity 178 also includes the proximal portion 200 of the sealing-module insert 198 disposed therein in at least some states of the one-or-more states of the RICC insertion assembly 100 such as the ready-to-operate state or one or more operating states of the RICC insertion assembly 100.

The needle-hub receptacle 180 is configured to hold the needle hub 146 of the introducer needle 104 therein. Indeed, the needle-hub receptacle 180 includes the needle hub 146 inserted therein in the ready-to-operate state of the RICC insertion assembly 100. While not shown, the coupler 108 can include a needle-hub lock about the needle-hub receptacle 180 configured to lock the needle hub 146 in the needle-hub receptacle 180. Indeed, a pair of lock buttons (e.g., spring-loaded lock buttons) of the needle-hub lock can be distributed between opposite sides of the coupler housing 172 such that each lock button of the lock buttons extends through the coupler housing 172 on its respective side of the coupler 108. Such lock buttons can be configured to unlock the needle hub 146 when the lock buttons are pressed into the coupler housing 172 for withdrawal of the introducer needle 104 from the coupler 108. Unlocking the lock buttons can immediately release the axial compression from the distal portion of the needle hub 146 compressing the proximal portion 200 of the sealing-module insert 198 in the sealing-module cavity 178. This allows the proximal and distal portions 200 and 202 of the sealing-module insert 198 to relax for withdrawing the introducer needle 104 from the coupler 108. This also allows the proximal and distal potions of the sealing-module insert 198 to be separated for the escape of the access guidewire 106 when the introducer needle 104 is withdrawn from the coupler 108 in the introducer needle-withdrawing step of the method set forth below.

The coupler-housing slot 182 formed along the length of the coupler housing 172 is configured to allow the access guidewire 106 to escape from the coupler housing 172 after the introducer needle 104 is withdrawn from the coupler 108 in the introducer needle-withdrawing step of the method set forth below. Indeed, as the introducer needle 104 is withdrawn from the coupler 108 in the introducer needle-withdrawing step, the proximal portion 200 of the sealing-module insert 198, too, is withdrawn from the sealing-module cavity 178 allowing the access guidewire 106 to escape from the coupler housing 172 once the proximal portion 200 of the sealing-module insert 198 is removed from the sealing-module cavity 178.

The access guidewire-connecting side arm 174 extends from the coupler 108 or the coupler housing 172 thereof. The access guidewire-connecting side arm 174 includes a connector 188 configured to connect to the access-guidewire hub 218 about the proximal-end portion of the access guidewire 106, which access-guidewire hub 218 extends from the proximal end of the RICC 102 in at least the ready-to-operate state of the RICC insertion assembly 100. While in the ready-to-operate state of the RICC insertion assembly 100, the distal portion of the access guidewire 106 is disposed in the needle shaft 144 and the access-guidewire hub 218 is connected to the connector 188 of the access guidewire-connecting side arm 174, thereby enforcing the loop 110 in the access guidewire 106 over which loop 110 the RICC 102 is disposed. When both the keeper 220 and the splittable casing-holding side arm 176 are present in the RICC insertion assembly 100, a distal portion of the splittable casing 222 is also held in the primary channel 190 of the splittable casing-holding side arm 176, thereby further enforcing the loop 110 in the access guidewire 106 over which loop 110 the RICC 102 is disposed.

When present, the splittable casing-holding side arm 176 extends from the coupler 108 or the coupler housing 172 thereof opposite the access guidewire-connecting side arm 174. The splittable casing-holding side arm 176 includes a primary channel 190 and a secondary channel 192. The primary channel 190 is configured to slidably hold the splittable casing 222 or the longitudinal composite of the splittable casing 222 and at least the access guidewire 106 therein. The secondary channel 192 is configured to guide the access guidewire 106 split away from the splittable casing 222 into the coupler 108, the splittable sealing module 196 thereof, and the needle shaft 144 sealed therein by way of the needle slot 150. A divergent point 194 of the splittable casing-holding side arm 176 between the primary channel 190 and the secondary channel 192 is configured to split the access guidewire 106 away from the splittable casing 222 as the longitudinal composite of the splittable casing 222 and at least the access guidewire 106 therein is pushed therein.

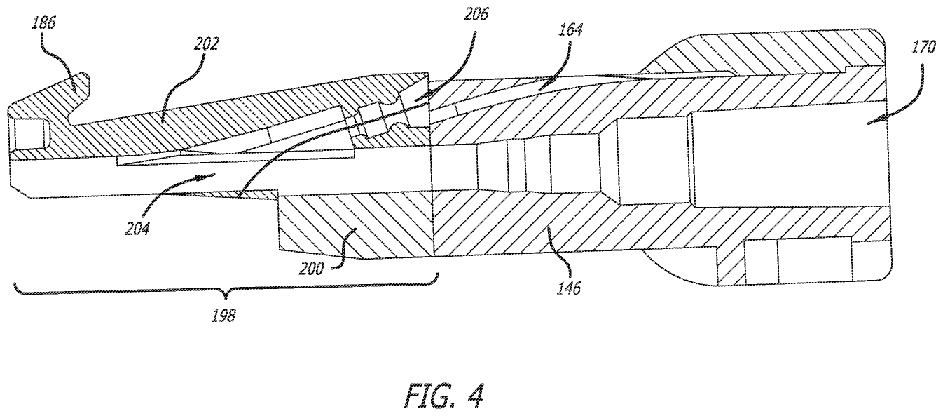

FIG. 4 illustrates a longitudinal cross section of a splittable sealing module 196 in accordance with some embodiments.

The splittable sealing module 196 of the RICC insertion assembly 100 includes the sealing-module cavity 178 of the coupler housing 172 and an elastomeric sealing-module insert 198 disposed therein, which sealing-module insert 198 is split between a captive proximal portion 200 and a removable distal portion 202 referred to herein as the proximal portion 200 of the sealing-module insert 198 and the distal portion 202 of the sealing-module insert 198, respectively. (Notably, the proximal and distal portions 200 and 202 of the sealing-module insert 198 can be formed of a same elastomer [e.g., silicone] or different elastomers.) The proximal portion 200 of the sealing-module insert 198 is configured to be disposed in or otherwise inserted into the sealing-module cavity 178 of the coupler housing 172 where the proximal portion 200 of the sealing-module insert 198 combines with the distal portion 202 of the sealing-module insert 198 to complete a pair of passageways through the splittable sealing module 196. By way of the foregoing passageways, the splittable sealing module 196 is configured to separately seal around the introducer needle 104 and the access guidewire 106 when the proximal and distal portions 200 and 202 of the sealing-module insert 198 are compressed in the sealing-module cavity 178 in the one-or-more states of the RICC insertion assembly 100 (e.g., the ready-to-operate state or the one-or-more operating states of the RICC insertion assembly 100), which allows the syringe 112 is able to aspirate blood in accordance with the blood-aspirating step of the method set forth below.

The pair of passageways completed when the proximal and distal portions 200 and 202 of the sealing-module insert 198 are combined in the sealing-module cavity 178 of the coupler housing 172 includes an introducer-needle passageway 204 and an access-guidewire passageway 206. As shown in FIG. 4, a proximal portion of the introducer-needle passageway 204 can be present in the proximal portion 200 of the sealing-module insert 198, but a distal portion of the introducer-needle passageway 204 is completed by the proximal and distal portions 200 and 202 of the sealing-module insert 198 when combined in the sealing-module cavity 178 of the coupler housing 172. As further shown in FIG. 4, an entirety of the access-guidewire passageway 206 can be completed by the proximal and distal portions 200 and 202 of the sealing-module insert 198 when combined in the sealing-module cavity 178 of the coupler housing 172. While other configurations of the sealing-module insert 198 are possible for providing the foregoing passageways, the splittable sealing module 196 is configured by way of the introducer-needle passageway 204 and the access-guidewire passageway 206 to separately seal around the proximal portion of the introducer needle 104 and the distal portion of the access guidewire 106, respectively, when the proximal and distal portions 200 and 202 of the sealing-module insert 198 are compressed in the sealing-module cavity 178 in the one-or-more states of the RICC insertion assembly 100. Notably, as opposed to the introducer-needle passageway 204, which passes through both proximal and distal ends of the sealing-module insert 198, a distal end of the access-guidewire passageway 206 connects to a medial portion of the introducer-needle passageway 204, thereby allowing the distal portion of the access guidewire 106 to be disposed in the needle shaft 144 while the distal portion of the access guidewire 106 is disposed in the splittable sealing module 196. Indeed, the access-guidewire passageway 206 is configured to direct the access guidewire 106 from the access-guidewire channel 164 of the needle hub 146 into both the sheath opening 162 of the sheath 142 and the needle slot 150 of the needle shaft 144 thereunder such that the access guidewire 106 can be disposed in the needle shaft 144 with the distal end of the access guidewire 106 just proximal of the needle tip 148 in the ready-to-operate state of the RICC insertion assembly 100.

The splittable sealing module 196 is configured to separately seal around the proximal portion of the introducer needle 104 and the distal portion of the access guidewire 106 when the sealing-module insert 198 is compressed in the sealing-module cavity 178. Indeed, the proximal and distal portions 200 and 202 of the sealing-module insert 198 can be radially compressed in the sealing-module cavity 178 or both axially and radially compressed in the sealing-module cavity 178 to seal around the introducer needle 104 and the access guidewire 106. With seals around the introducer needle 104 and the access guidewire 106 in accordance with the foregoing, the syringe 112 is able to aspirate blood in accordance with the blood-aspirating step of the method set forth below.

In an example of radial compression of the sealing-module insert 198 in the sealing-module cavity 178, the proximal and distal portions 200 and 202 of the sealing-module insert 198 can be radially compressed in the sealing-module cavity 178 by the coupler housing 172 itself. Without limiting such embodiments, two halves of the coupler housing 172 can be coupled together like a clamshell by way of a hinge opposite the coupler-housing slot 182 formed between the two halves of the coupler housing 172. One or more clamps across the coupler-housing slot 182 can be clamped in at least the ready-to-operate state of the RICC insertion assembly 100 to hold the two halves of the coupler housing 172 together, apply sufficient pressure to radially compress the proximal and distal portions 200 and 202 of the sealing-module insert 198 in the sealing-module cavity 178, and seal the sealing-module insert 198 around the introducer needle 104 and the access guidewire 106. The one-or-more clamps can be unclamped in the one-or-more operating states of the RICC insertion assembly 100 to relieve the radial compression and allow the introducer needle 104 to be withdrawn from the coupler 108 for the escape of the access guidewire 106 through the coupler-housing slot 182 thereafter.

In an example of both radial and axial compression of the sealing-module insert 198 in the sealing-module cavity 178, the proximal and distal portions 200 and 202 of the sealing-module insert 198 can be both axially and radially compressed in the sealing-module cavity 178 by way of the distal portion of the needle hub 146. The distal portion of the needle hub 146 axially compresses the proximal and distal portions 200 and 202 of the sealing-module insert 198 in the sealing-module cavity 178 when the needle hub 146 is disposed in the needle-hub receptacle 180 such as in the ready-to-operate state of the RICC insertion assembly 100. Axial compression of the proximal and distal portions 200 and 202 of the sealing-module insert 198 in the sealing-module cavity 178, in turn, radially compresses the proximal and distal portions 200 and 202 of the sealing-module insert 198 in the sealing-module cavity 178, thereby sealing the sealing-module insert 198 around the introducer needle 104 and the access guidewire 106. The needle hub 146 can be removed from the needle-hub receptable in the one-or-more operating states of the RICC insertion assembly 100 to relieve both the axial and the radial compression and allow the introducer needle 104 to be withdrawn from the coupler 108 for the escape of the access guidewire 106 through the coupler-housing slot 182 thereafter.

The coupler housing 172 of the coupler 108 can further include a blade 212 extending into the introducer-needle passageway 204 of the splittable sealing module 196. The blade 212 can be overmolded into the distal portion 202 of the sealing-module insert 198, thereby integrating the blade 212 therein. The blade 212 or a blade tip 214 thereof can be disposed in the needle slot 150 under a distal end of the sheath opening 162 in at least the ready-to-operate state of the RICC insertion assembly 100. The blade 212 includes a distal-facing blade edge 216 configured to cut the sheath 142 away from the needle shaft 144 as the introducer needle 104 is withdrawn from the coupler 108 through the introducer-needle passageway 204 in the introducer needle-withdrawing step of the method set forth below. Cutting the sheath 142 away from the needle shaft 144 as the introducer needle 104 is withdrawn from the coupler 108 allows the access guidewire 106 to escape from the needle shaft 144 by way of the needle slot 150.

FIGS. 1 and 3 illustrate different views of the access guidewire 106 of the RICC insertion assembly 100 in accordance with some embodiments.

The access guidewire 106 includes a proximal portion including a proximal end and a distal portion including a distal end. In the ready-to-operate state of the RICC insertion assembly 100, the proximal end of the access guidewire 106 is coupled to the access guidewire-connecting side arm 174 by way of an access-guidewire hub 218 about a proximal-end portion of the access guidewire 106 that includes the distal end. In addition, the proximal portion of the access guidewire 106 extends along the primary lumen 130 of the RICC 102. The distal portion of the access guidewire 106 also extends along the primary lumen 130 of the RICC 102, but the distal portion of the access guidewire 106 further extends out the distal end of the RICC 102 as an extracatheteral portion of the access guidewire 106, into the splittable sealing module 196 over the needle hub 146 by way of the access-guidewire channel 164, into the needle shaft 144 through both the sheath opening 162 of the sheath 142 and the needle slot 150 of the needle shaft 144, and along the needle lumen 158 of the introducer needle 104 in the ready-to-operate state of the RICC insertion assembly 100. As shown in FIG. 1, the distal end of the access guidewire 106 is disposed in the needle lumen 158 just proximal of the needle tip 148 in the ready-to-operate state of the RICC insertion assembly 100. Again, the proximal and distal ends of the access guidewire 106 enforce the loop 110 in the access guidewire 106 in the ready-to-operate state of the RICC insertion assembly 100, which loop 110 the RICC 102 is disposed over, thereby keeping the RICC insertion assembly 100 in a relatively compact form.

The access guidewire 106 can include a guidewire tip in the distal portion of the access guidewire 106, which adopts a T shape configured to prevent puncturing a back wall of a blood vessel. Such a guidewire tip assumes a straightened state in the ready-to-operate state of the RICC insertion assembly 100 and a curved state when the guidewire tip is advanced beyond the needle tip 148 (e.g., advanced into a blood-vessel lumen) in the one-or-more operating states of the RICC insertion assembly 100 in which the access guidewire 106 is deployed.

The access guidewire 106 can further include a bare-wire portion and a wound-wire portion distal of the bare-wire portion, proximal of the bare-wire portion, or both. While not shown, the bare-wire portion, when present, distally extends through the access-guidewire passageway 206 of the splittable sealing module 196 in at least the ready-to-operate state of the RICC insertion assembly 100 such that the splittable sealing module 196 forms a fluid-tight seal around the bare-wire portion of the access guidewire 106. Notably, the foregoing bare-wire portion can instead be a flat-wound or ground-wound portion of the access guidewire 106, wherein the flat-wound portion includes windings of a tape instead of a round wire, and wherein the ground-wound portion includes windings of a round wire ground down to flatten the windings.

FIG. 1 illustrate a keeper 220 of the RICC insertion assembly 100 in accordance with some embodiments.

As shown, the keeper 220 can include a splittable casing 222 and a catheter-hub holder 224 to which a proximal end of the splittable casing 222 is attached.

The splittable casing 222 can form a longitudinal composite with the catheter tube 114, the access guidewire 106, or both the catheter tube 114 and the access guidewire 106 in the RICC insertion assembly 100. With respect to the RICC insertion assembly 100 of FIG. 1, for example, the splittable casing 222 is over and, thusly, forms the longitudinal composite with both the catheter tube 114 and the access guidewire 106 in a portion of the RICC insertion assembly 100 nearest the catheter-hub holder 224, in which portion the access guidewire 106 is disposed in the primary lumen 130 of the RICC 102. Further with respect to the RICC insertion assembly 100 of FIG. 1, the splittable casing 222 is over and, thusly, forms the longitudinal composite with just the access guidewire 106 in a portion of the RICC insertion assembly 100 nearest the coupler 108 or the splittable casing-holding side arm 176 thereof, in which portion the extracatheteral portion of the access guidewire 106 extends from the distal end of the RICC 102. The splittable casing 222 is configured to split along its length, for example, as it is slid over the divergent point 194 of the splittable casing-holding side arm 176 of the coupler 108, such that the extracatheteral portion of the access guidewire 106 is initially revealed and the distal portion of the catheter tube 114 is subsequently revealed. In this way, the splittable casing 222 is configured to keep the catheter tube 114 and at least the distal portion of the access guidewire 106 sterile until deployed.

The catheter-hub holder 224 is configured to hold the catheter hub 116 therein as well as keep the splittable casing 222 in position over the catheter tube 114 and the access guidewire 106, particularly the extracatheteral portion of the access guidewire 106. The catheter-hub holder 224 includes a perimetrical wall 226 around at least a portion (e.g., a proximal portion) of a perimeter of the catheter-hub holder 224. The perimetrical wall 226 defines a recess into which the catheter hub 116 fits with an engineering fit (e.g., a clearance fit such as a running, sliding, or location fit or a transition fit such as a similar or fixed fit as classified by the International Organization for Standardization [“ISO”]) as well as one or more gaps for extension of the one-or-more extension legs 118 therethrough. Additionally or alternatively, the catheter-hub holder 224 can include a wing corresponding to a suture wing of the catheter hub 116. Such a wing can include posts configured to insert into suture-wing holes of the suture wing of the catheter hub 116 with an engineering fit.

Methods

Methods of the introducer needle 104 and the RICC insertion assembly 100 include at least a method for making the introducer needle 104 and a method for inserting the RICC 102 into a blood-vessel lumen of a patient, respectively.

The method for making the introducer needle 104 includes one or more steps selected from a metal strip-rolling step, a seam-welding step, a cold-working step, a grinding step, a needle-slot creating step, a wall-bending step, an edge-finishing step, a sheath-disposing step, a sheath opening-creating step, and a needle hub-fixing step.

The metal strip-rolling step includes rolling a strip of metal such as stainless steel into a metal tube. For example, the metal strip-rolling step can include rolling the strip of metal into the metal tube with a milling machine or stamping press. Such a metal tube can include a longitudinal seam formed between edges of longitudinal sides of the strip of metal. However, such a metal tube can alternatively include the needle slot 150 formed between the edges of the longitudinal sides of the strip of metal instead of the longitudinal seam. If the metal tube is formed with the needle slot 150 at an appropriate gauge (e.g., 18 G) in the metal strip-rolling step, the grinding step can be performed following the metal strip-rolling step to make the needle shaft 144 without any additional post processing such as the edge-finishing step. (See, for example, the needle shaft 144 of FIG. 7A.) Notably, such a needle shaft has a comparable flexural strength to that formed following, for example, the seam-welding step, the cold-working step, the needle slot-creating step, and the edge-finishing step.

The seam-welding step includes welding the seam formed between the edges of the sides of the strip of metal. For example, the seam-welding step can include laser welding the seam formed between the edges of the sides of the strip of metal.

The cold-working step includes pushing the metal tube through one or more dies one or more times, thereby reducing an outer diameter of the metal tube (e.g., 18 G) while simultaneously increasing a thickness of a metal-tube wall of the metal tube. Subsequent to the cold-working step, the metal tube can be scored and broken into two or more smaller metal tubes and batched together for the grinding step; however, for expository convenience, such further processing is described with reference to the foregoing metal tube.

The grinding step includes grinding an end of the metal tube at a plurality of angles to form the needle shaft 144 with the needle tip 148 having the bevel. For example, grinding the end of the metal tube at a first angle forms the primary bevel 154 including the heel of the bevel; grinding the end of the metal tube at two similar, but opposite second angles forms the tip bevel 152 of the bevel.

The needle-slot creating step includes creating the needle slot 150 in the needle shaft 144. In an example, the needle-slot creating step can include cutting the needle slot 150 into the needle shaft 144 by way of machining or laser cutting using, for example a computer numerical control (“CNC”) laser cutting machine, which can be useful for cutting and turning the needle shaft 144 during the cutting to form the needle slot 150 when non-linear such as at least partially helical as set forth above. The opposite needle-slot walls 230 that result from the cutting of the needle slot 150 into the needle shaft 144 face each other and are parallel to each other. In addition, the needle-slot walls 230 are opposite the bottom of the needle slot 150. In another example, the needle-slot creating step can include grinding the needle slot 150 into the needle shaft 144. The opposite needle-slot walls 230 that result from the grinding of the needle slot 150 into the needle shaft 144 face away from the bottom of the needle slot 150. As set forth above, the needle slot 150 extends from the proximal portion of the needle shaft 144 through the needle tip 148, which needle tip 148 includes the bevel having the tip bevel 152 and the heel.

The wall-bending step includes bending the needle-slot walls 230 in toward the bottom of the needle slot 150, thereby increasing the flexural strength of the needle shaft 144.

The edge-finishing step includes finishing (e.g., grinding, polishing, etc.) edges of the needle-slot walls 230, thereby minimizing or eliminating sharp, access guidewire-fraying edges or burrs of the needle slot 150.

The sheath-disposing step includes disposing the sheath 142 over the needle shaft 144, which sheath 142 seals the needle slot 150 thereunder. The disposing of the sheath 142 over the needle shaft 144 can include inserting the needle shaft 144 into the sheath 142 and heat shrinking the sheath 142 over the needle shaft 144 to seal the needle slot 150 thereunder.

The sheath opening-creating step includes creating the sheath opening 162 in the sheath 142. The creating of the sheath opening 162 in the sheath 142 can include cutting (e.g., laser cutting) the sheath opening 162 into the needle shaft 144 after the disposing of the sheath 142 over the needle shaft 144 in the sheath-disposing step. As set forth above, the sheath 142 seals the needle slot 150 thereunder but for the sheath opening 162 in the proximal portion of the sheath 142.

The needle hub-fixing step includes fixing the needle hub 146 around both the proximal portion of the needle shaft 144 and the proximal portion of the sheath 142, thereby forming the introducer needle 104. The needle hub-fixing step can include pressing both the proximal portion of the needle shaft 144 and the proximal portion of the sheath 142 into the needle hub 146 with an engineering fit selected from a transition and an interference fit. Additionally or alternatively, the needle hub-fixing step can include adhering the needle hub 146 to the both the proximal portion of the needle shaft 144 and the proximal portion of the sheath 142.

Adverting to the method for inserting the RICC 102 into the blood-vessel lumen of the patient, the method can include one or more steps selected from an insertion assembly-obtaining step, a state-confirming step, a needle tract-establishing step, a blood-aspirating step, an access guidewire-advancing step, a sheath-splitting step, an introducer needle-withdrawing step, a RICC-advancing step, an access guidewire-withdrawing step, a maneuver guidewire-advancing step, another RICC-advancing step, and a maneuver guidewire-withdrawing step.

The insertion assembly-obtaining step includes obtaining the RICC insertion assembly 100, optionally, already in the ready-to-operate state thereof. As set forth above, the RICC insertion assembly 100 includes the RICC 102, the introducer needle 104, the access guidewire 106, and the coupler 108 coupling the RICC 102 and the introducer needle 104 together. In addition, the RICC insertion assembly 100 includes the splittable sealing module 196 formed between at least the sealing-module cavity 178 of the coupler housing 172 of the coupler 108, the distal portion 202 of the sealing-module insert 198 disposed in the sealing-module cavity 178, and the proximal portion 200 of the sealing-module insert 198 coupled to the distal portion of the needle hub 146 of the introducer needle 104. The splittable sealing module 196 separately seals around the proximal portion of the introducer needle 104 and the distal portion of the access guidewire 106 in at least the ready-to-operate state of the RICC insertion assembly 100. Within the splittable sealing module 196, the distal portion of the access guidewire 106 is disposed in the needle shaft 144 through the needle slot 150. This is accomplished through a connection between the distal end of the access-guidewire passageway 206 and the medial portion of the introducer-needle passageway 204 in the sealing-module insert 198.

The state-confirming step includes confirming the RICC insertion assembly 100 is in the ready-to-operate state thereof before the needle tract-establishing step. Such confirmation ensures the proximal and distal portions 200 and 202 of the sealing-module insert 198 are compressed in the sealing-module cavity 178 sealing the introducer-needle passageway 204 and the access-guidewire passageway 206 around the introducer needle 104 and access guidewire 106, respectively. Notably, if the RICC insertion assembly 100 is not in the ready-to-operate state upon obtaining it in the insertion assembly-obtaining step, the RICC insertion assembly 100 can be adjusted to put it in the ready-to-operate state for subsequent steps.

The needle tract-establishing step includes establishing a needle tract from an area of skin to the blood-vessel lumen with the introducer needle 104. The needle tract-establishing step can also include ensuring blood flashes back into the introducer needle 104 (e.g., the needle hub 146 of the introducer needle 104), the syringe 112 (e.g., the syringe tip, a barrel of the syringe 112, or both) fluidly connected to the introducer needle 104, or both the introducer needle 104 and the syringe 112, thereby confirming the needle tract extends into the blood-vessel lumen. To enhance blood flashback, a slight vacuum can be drawn with the syringe 112 while establishing the needle tract.

The blood-aspirating step includes aspirating blood with the syringe 112 to confirm the needle tract extends into the blood-vessel lumen before withdrawing the introducer needle 104 from the coupler 108 in the introducer needle-withdrawing step. Again, the sheath 142 over the needle shaft 144 seals the needle slot 150 of the needle shaft 144 thereunder. In particular, the sheath 142 seals the needle slot 150 outside of the splittable sealing module 196. The splittable sealing module 196, in turn, seals over the sheath opening 162 of the sheath 142, which sheath opening 162 allows the access guidewire 106 to pass from the access-guidewire passageway 206 formed between the of the proximal and distal portions 200 and 202 of the sealing-module insert 198 and into the needle shaft 144 by way of the needle slot 150. The splittable sealing module 196 also seals around the distal portion of the access guidewire 106. Such seals enable the syringe 112 to aspirate blood in the blood-aspirating step.

The access guidewire-advancing step includes advancing the distal portion of the access guidewire 106 through both the splittable sealing module 196 and the needle slot 150 of the needle shaft 144 such that the distal end of the access guidewire 106 is advanced from its initial location in the introducer needle 104 into the blood-vessel lumen. As set for the above, the initial location of the distal end of the access guidewire 106 is just proximal of the needle tip 148 when the RICC insertion assembly 100 is in the ready-to-operate state of the RICC insertion assembly 100.

When the RICC insertion assembly 100 does not include the keeper 220 and the splittable casing 222 over both the catheter tube 114 of the RICC 102 and the extracatheteral portion of the access guidewire 106, the access guidewire-advancing step includes pinching the extracatheteral portion of the access guidewire 106 extending from the distal end of the RICC 102 and pushing the access guidewire 106 into the needle shaft 144 via the splittable sealing module 196 of the coupler 108. Advancing the access guidewire 106 in this way reduces the loop 110 in the access guidewire 106 over which loop 110 the RICC 102 is disposed. As set forth above, the loop 110 is enforced by the distal portion of the access guidewire 106 disposed in the needle shaft 144 and the access-guidewire hub 218 of the access guidewire 106 connected to the connector 188 of the access guidewire-connecting side arm 174 of the coupler 108.

When the RICC insertion assembly 100 includes the keeper 220 and the splittable casing 222 over both the catheter tube 114 of the RICC 102 and the extracatheteral portion of the access guidewire 106, the access guidewire-advancing step includes pinching the splittable casing 222 over both the catheter tube 114 of the RICC 102 and the extracatheteral portion of the access guidewire 106 extending from the distal end of the RICC 102. The access guidewire-advancing step also includes pushing the splittable casing 222 into the primary channel 190 of the splittable casing-holding side arm 176 of the coupler 108 while pinching the splittable casing 222. With such pinching and pushing of the splittable casing 222 into the primary channel 190 of the splittable casing-holding side arm 176, the access guidewire 106 splits away from the splittable casing 222 over the divergent point 194 of the splittable casing-holding side arm 176 between the primary channel 190 and the secondary channel 192, into the secondary channel 192 of the splittable casing-holding side arm 176, and into the needle shaft 144 via the splittable sealing module 196 of the coupler 108. Advancing the access guidewire 106 in this way reduces the loop 110 in the access guidewire 106 over which loop 110 the RICC 102 and the splittable casing 222 are disposed. The loop 110 is enforced by at least the splittable casing 222 held in the primary channel 190 of the splittable casing-holding side arm 176 and the access-guidewire hub 218 of the access guidewire 106 connected to the connector 188 of the access guidewire-connecting side arm 174 of the coupler 108.

The introducer needle-withdrawing step includes withdrawing the introducer needle 104 from the coupler 108 before the RICC-advancing step, thereby leaving the access guidewire 106 in place in the blood-vessel lumen. The withdrawing of the introducer needle 104 from the coupler 108 removes the needle hub 146 of the introducer needle 104 from the needle-hub receptacle 180 and, with the needle hub 146, the proximal portion 200 of the sealing-module insert 198 from the sealing-module cavity 178, thereby splitting the proximal and distal portions 200 and 202 of the sealing-module insert 198 away from each other and unsealing the proximal portion of the introducer needle 104 and the distal portion of the access guidewire 106 so the access guidewire 106 is allowed to escape the splittable sealing module 196.

The introducer needle-withdrawing step can also include cutting the needle slot-sealing sheath 142 of the introducer needle 104 away from the needle shaft 144. As set forth above, the coupler housing 172 of the coupler 108 can include the blade 212 extending into the splittable sealing module 196 such that the blade 212 is disposed in the needle slot 150 under the distal end of the sheath opening 162 of the sheath 142 with the distal-facing blade edge 216 for the cutting of the sheath 142 away from the needle shaft 144. Should the coupler housing 172 not include the blade 212, the method can further include the sheath-splitting step, which includes splitting the sheath 142. The cutting or splitting of the sheath 142 away from the needle shaft 144 allows the access guidewire 106 to escape from the needle shaft 144 by way of the needle slot 150, which needle slot 150 can vary as set forth above. In addition, the coupler-housing slot 182 of the coupler housing 172 allows the access guidewire 106 to escape from the coupler housing 172 after the withdrawing of the introducer needle 104 from the coupler 108.

The RICC-advancing step includes advancing the catheter tube 114 of the RICC 102 over the access guidewire 106 and into the blood-vessel lumen, thereby inserting the RICC 102 into the blood-vessel lumen.

The access guidewire-withdrawing step includes withdrawing the access guidewire 106 leaving the catheter tube 114 in place in the blood-vessel lumen.

The maneuver guidewire-advancing step includes advancing a maneuver guidewire into the blood-vessel lumen by way of the primary lumen 130 of the RICC 102 and to a lower ⅓ of an SVC of a heart of the patient.

The other RICC-advancing step includes advancing the catheter tube 114 farther into the blood-vessel lumen over the maneuver guidewire to the lower ⅓ of the SVC of the heart of the patient.

The maneuver guidewire-withdrawing step includes withdrawing the maneuver guidewire leaving the catheter tube 114 in place in the lower ⅓ of the SVC.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

1. An introducer needle, comprising:
a needle shaft including a needle slot extending from a proximal portion of the needle shaft through a distal needle tip having a bevel with a tip bevel and a heel, the needle shaft including one or more features selected from a group of features consisting of:
a) opposite needle-slot walls face each other and are parallel to each other consistent with the needle slot being cut into the needle shaft;
b) the opposite needle-slot walls face each other but are oblique to each other consistent with the needle shaft being stamped or rolled to form the needle slot;
c) the opposite needle-slot walls face away from a bottom of the needle slot consistent with the needle slot being ground into the needle shaft;
d) the opposite needle-slot walls face toward the bottom of the needle slot consistent with opposite needle-slot walls being bent in toward the bottom of the needle slot; and
e) the opposite needle-shaft walls are parallel to each other consistent with the needle shaft being stamped or rolled to form the needle slot;
a sheath over the needle shaft sealing the needle slot thereunder but for a sheath opening in a proximal portion of the sheath; and
a needle hub around both the proximal portion of the needle shaft and a proximal portion of the sheath.

2. The introducer needle of claim 1, wherein the needle slot extends along at least a portion of a top of the needle shaft, the top of the needle shaft including the heel of the bevel.

3. The introducer needle of claim 1, wherein the needle slot extends along at least a portion of a bottom of the needle shaft, the bottom of the needle shaft including the tip bevel of the bevel.

4. The introducer needle of claim 1, wherein the needle slot extends along at least a portion of a side of the needle shaft, the side of the needle shaft between the heel and tip bevel of the bevel.

5. The introducer needle of claim 1, wherein the needle slot bisects the heel of the bevel.

6. The introducer needle of claim 1, wherein the needle slot bisects the tip bevel of the bevel.

7. The introducer needle of claim 1, wherein the needle slot intersects the heel, the tip bevel, or both the heel and the tip bevel of the bevel without bisecting the heel or tip bevel of the bevel.

8. The introducer needle of claim 1, wherein the needle slot is linear from the proximal portion of the needle shaft through the needle tip.

9. The introducer needle of claim 1, wherein the needle slot is non-linear from the proximal portion of the needle shaft through the needle tip.

10. The introducer needle of claim 9, wherein the needle slot is at least partially helical, the needle slot forming at least part of a helix between the proximal portion of the needle shaft and the needle tip.

11. The introducer needle of claim 1, wherein the needle slot has a constant needle-slot width.

12. The introducer needle of claim 1, wherein the needle slot has a non-constant needle-slot width.

13. The introducer needle of claim 12, wherein the needle slot includes one or more needle-slot openings wider than a remainder of the needle slot.

14. The introducer needle of claim 12, wherein the needle slot includes a taper, a reverse taper, or a stepwise change in the needle-slot width from the proximal portion of the needle shaft through the needle tip.

15. The introducer needle of claim 1, wherein the needle slot is short of a proximal end of the needle shaft.

16. The introducer needle of claim 1, wherein the needle slot extends through a proximal end of the needle shaft.

17. The introducer needle of claim 1, wherein edges of the needle-slot walls are finished, thereby minimizing or eliminating sharp, access guidewire-fraying edges of the needle slot.

18. A rapidly insertable central catheter (“RICC”) insertion assembly, comprising:
a RICC including a catheter tube and a primary lumen therethrough;
an introducer needle including:
a needle shaft including a needle slot extending from a proximal portion of the needle shaft through a distal needle tip having a bevel with a tip bevel and a heel, the needle shaft including one or more features selected from a group of features consisting of:
a) opposite needle-slot walls face each other and are parallel to each other consistent with the needle slot being cut into the needle shaft;
b) the opposite needle-slot walls face each other but are oblique to each other consistent with the needle shaft being stamped or rolled to form the needle slot;
c) the opposite needle-slot walls face away from a bottom of the needle slot consistent with the needle slot being ground into the needle shaft;
d) the opposite needle-slot walls face toward the bottom of the needle slot consistent with opposite needle-slot walls being bent in toward the bottom of the needle slot; and
e) the opposite needle-shaft walls are parallel to each other consistent with the needle shaft being stamped or rolled to form the needle slot;
a sheath over the needle shaft sealing the needle slot thereunder but for a sheath opening in a proximal portion of the sheath; and
a needle hub around both the proximal portion of the needle shaft and a proximal portion of the sheath;
an access guidewire including a proximal portion disposed in the primary lumen of the RICC and a distal portion disposed in the needle shaft through both the sheath opening and needle slot; and
a coupler coupling the RICC and the introducer needle together.

19. The RICC insertion assembly of claim 18, wherein the needle slot extends along at least a portion of a top of the needle shaft, the top of the needle shaft including the heel of the bevel.

20. The RICC insertion assembly of claim 18, wherein the needle slot extends along at least a portion of a bottom of the needle shaft, the bottom of the needle shaft including the tip bevel of the bevel;

21. The RICC insertion assembly of claim 18, wherein the needle slot extends along at least a portion of a side of the needle shaft, the side of the needle shaft between the heel and tip bevel of the bevel.

22. The RICC insertion assembly of claim 18, wherein the needle slot bisects the heel of the bevel.

23. The RICC insertion assembly of claim 18, wherein the needle slot bisects the tip bevel of the bevel.

24. The RICC insertion assembly of claim 18, wherein the needle slot intersects the heel, the tip bevel, or both the heel and the tip bevel of the bevel without bisecting the heel or tip bevel of the bevel.

25. The RICC insertion assembly of claim 18, wherein the needle slot is linear from the proximal portion of the needle shaft through the needle tip.

26. The RICC insertion assembly of claim 18, wherein the needle slot is non-linear from the proximal portion of the needle shaft through the needle tip.

27. The RICC insertion assembly of claim 26, wherein the needle slot is at least partially helical, the needle slot forming at least part of a helix between the proximal portion of the needle shaft and the needle tip.

28. The RICC insertion assembly of claim 18, wherein the needle slot has a constant needle-slot width.

29. The RICC insertion assembly of claim 18, wherein the needle slot has a non-constant needle-slot width.

30. The RICC insertion assembly of claim 29, wherein the needle slot includes one or more needle-slot openings wider than a remainder of the needle slot.

31. The RICC insertion assembly of claim 29, wherein the needle slot includes a taper, a reverse taper, or a stepwise change in the needle-slot width from the proximal portion of the needle shaft through the needle tip.

32. The RICC insertion assembly of claim 18, wherein the needle slot is short of a proximal end of the needle shaft.

33. The RICC insertion assembly of claim 18, wherein the needle slot extends through a proximal end of the needle shaft.

34. The RICC insertion assembly of claim 18, wherein edges of the needle-slot walls are finished, thereby minimizing or eliminating sharp, access guidewire-fraying edges of the needle slot.

35. The RICC insertion assembly of claim 18, the coupler including:
a coupler housing including a sealing-module cavity; and
a sealing-module insert disposed in the sealing-module cavity, the sealing-module cavity and the sealing-module insert forming a sealing module of the RICC insertion assembly configured to separately seal around a proximal portion of the introducer needle including the sheath opening and the distal portion of the access guidewire when compressed in the sealing-module cavity.

36. The RICC insertion assembly of claim 35, wherein the coupler housing includes a longitudinal coupler-housing slot configured to allow the access guidewire to escape from the coupler housing after a distal portion of the sealing-module insert is removed from the sealing-module cavity with withdrawal of the introducer needle from the coupler.

37. The RICC insertion assembly of claim 35, wherein the coupler includes a blade extending into the sealing module such that the blade is disposed in the needle slot under a distal end of the sheath opening, the blade including a distal-facing blade edge configured to cut the sheath away from the needle shaft as the introducer needle is withdrawn from the coupler, thereby allowing the access guidewire to escape from the needle shaft by way of the needle slot.

38-51. (canceled)
